(19) United States
(12) Patent Application Publication
(10) Pub. No.: US 2013/0059003 A1
(43) Pub. Date: Mar. 7, 2013

(54) SUSTAINED RELEASE DONEPEZIL FORMULATIONS

(71) Applicants: Snehalatha Movva, Hyderabad (IN); Hemanth Prakash Joshi, Masari Extension Gadag (IN); Rajan Kumar Verma, New Delhi (IN); Badri Narayanan Vishanathan, Chennai (IN); Lakshmi Prasanna Gubbala, Secunderabad (IN); Prasad Vure, Secunderabad (IN)

(72) Inventors: Snehalatha Movva, Hyderabad (IN); Hemanth Prakash Joshi, Masari Extension Gadag (IN); Rajan Kumar Verma, New Delhi (IN); Badri Narayanan Vishanathan, Chennai (IN); Lakshmi Prasanna Gubbala, Secunderabad (IN); Prasad Vure, Secunderabad (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/513,637

(22) PCT Filed: Dec. 3, 2010
(86) PCT No.: PCT/US10/58908

§ 371 Date: Nov. 15, 2012

Related U.S. Application Data
(60) Provisional application No. 61/314,387, filed on Mar. 16, 2010.

(30) Foreign Application Priority Data
Dec. 4, 2009 (IN) 2995/CHE/2009

(51) Int. Cl. A61K 9/22; A61P 25/00; A61P 25/28; A61K 31/445

(52) U.S. Cl. 424/465; 514/319

(57) ABSTRACT

Sustained release formulations comprising donepezil, or its pharmaceutically acceptable salts, and methods of preparing the sustained release formulations.

Aspects of the present disclosure provide sustained release formulations comprising donepezil or its pharmaceutically acceptable salts and methods of preparing the sustained release formulations. Further aspects of the disclosure include methods of using sustained release formulations in treating dementia of Alzheimer's disease.

The drug having the adopted name “donepezil” has a chemical name (±)-2,3-dihydro-5,6-dimethoxy-2-[[1-(phenylmethyl)-4-piperidinyl]methyl]-1H-inden-1-one. It has the empirical formula C₂₄H₂₉NO₃. Its salt, donepezil hydrochloride, is a white crystalline powder and is freely soluble in chloroform, soluble in water and glacial acetic acid, slightly soluble in ethanol and acetonitrile, and practically insoluble in ethyl acetate and n-hexane. The salt is represented by structural formula (I).

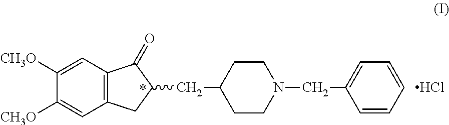

Donepezil is a centrally acting reversible acetyl cholinesterase inhibitor. Donepezil hydrochloride is the active ingredient in products sold as ARICEPT™ for oral administration, in film coated tablets containing 5, 10, or 23 mg of donepezil hydrochloride. Also available are ARICEPT™ ODT tablets for oral administration containing 5 or 10 mg of donepezil hydrochloride. ARICEPT products are indicated for the treatment of dementia of the Alzheimer's type.

Inactive ingredients in ARICEPT 5 mg and 10 mg tablets are lactose monohydrate, corn starch, microcrystalline cellulose, hydroxypropyl cellulose, and magnesium stearate. The film coating contains talc, polyethylene glycol, hypromellose, and titanium dioxide. Additionally, the 10 mg tablet contains yellow iron oxide (synthetic) as a coloring agent.

Inactive ingredients in ARICEPT 23 mg tablets include ethylcellulose, hydroxypropyl cellulose, lactose monohydrate, magnesium stearate, and methacrylic acid copolymer, Type C. The film coating includes ferric oxide, hypromellose 2910, polyethylene glycol 8000, talc and titanium dioxide.

The immediate release of cholinesterase inhibitors results in a spike in the patient's blood plasma levels within 2 to 5 hours after administration of the drug. Rogers et al., British Journal of Clinical Pharmacology, 46 (Suppl. 1), pages 1-6, 1998 describe the mean plasma concentration time curves following single dose administrations of 2 mg, 4 mg, and 6 mg of donepezil hydrochloride to groups of six healthy male volunteers. The patients administered 2 mg donepezil hydrochloride experienced a peak plasma concentration (Cₘₐₓ) of 3.2±0.6 ng/mL and the time at which the peak concentration occurred (tₘₐₓ) was 4.5±1.2 hours; the patients administered 4 mg of donepezil hydrochloride experienced a Cₘₐₓ of 6.9±0.7 ng/mL at a tₘₐₓ of 4.7±1.9 hours; and the patients administered 6 mg donepezil hydrochloride experienced a Cₘₐₓ of 11.6±2.8 ng/mL at a tₘₐₓ of 3.2±1.5 hours. The total areas under the plasma concentration-time curves (AUC_{t-∞}) for patients administered 2 mg, 4 mg, and 6 mg donepezil hydrochloride were 225.1±82.6 ng·hour/mL; 518.6±154.5 ng·hour/mL; and 706.6±195.8 ng·hour/mL, respectively.

According to the U.S. prescribing information for ARICEPT tablets, the 10 mg product gives a peak plasma concentration 3 hours after oral dosing, while the peak plasma concentration is achieved in about 8 hours for the 23 mg product.

Donepezil is disclosed in U.S. Pat. Nos. 4,895,841 and 5,100,901. U.S. Pat. Nos. 5,985,864, 6,140,321, and 6,245,911 disclose various polymorphic forms of donepezil or its salts. U.S. Pat. No. 6,372,760 discloses stabilized compositions of anti-dementia drugs. U.S. Patent Application Publication Nos. 2010/0152164, 2009/0208579, 2008/0213368, 2007/0129402, 2006/0280789, 2006/0159753, 2006/0246003, 2006/0160852, and 2005/0232990 disclose various sustained release formulations of anti-dementia drugs.

With the use of an acetyl cholinesterase inhibitor, patients may experience cholinergic adverse events when first dosed, especially at higher doses. The most common adverse events from ARICEPT cholinomimetic effects include nausea, diarrhea, insomnia, vomiting, muscle cramps, fatigue, bradycardia, abdominal pain, and anorexia, resulting in a reduction of patient compliance. These undesirable effects are due to the initial spike in blood plasma levels. Therefore, an initial therapeutic regimen is often recommended wherein donepezil is first introduced at low doses for several weeks followed by the gradual increase to the appropriate active dose for the patient.

A sustained release formulation may be advantageous in reducing the undesirable side effects associated with the rapid increase in blood plasma concentration levels immediately after administration of the drug. Such sustained release formulations could provide a uniform and constant rate of release over an extended period of time, which may achieve a stable and desired blood level of donepezil without the initial spike in drug plasma level.

However, a sustained release formulation of highly soluble drugs such as donepezil or its salts has been found to be difficult to formulate for several reasons. First, drugs that are soluble in water tend to generate a sustained release product susceptible to a phenomenon known as dose dumping. That is, release of the active ingredient is delayed for a time but, once release begins to occur, the rate of release is very high. Moreover, fluctuations tend to occur in the plasma concentrations of the active ingredient, which increase the likelihood of undesirable side effects. Further, some degree of diurnal variation in plasma concentration of the active ingredient has also been observed. Finally, it has been found to be difficult to achieve the desired dissolution profiles or to control the rate of release of the soluble medicament. The literature discloses some approaches for preparing sustained release formulations of donepezil. The literature also teaces the need for a water insoluble polymer for preparing sustained release formulations of donepezil.

### SUMMARY

Aspects of the present disclosure provide sustained release formulations comprising donepezil, or its pharmaceutically acceptable salts, and methods of preparing the sustained release formulations. Further aspects of the disclosure provide methods of using formulations of the disclosure to treat dementia of Alzheimer's disease.

In embodiments, the disclosure relates to sustained release formulations comprising donepezil or salts thereof, wherein the formulations, upon initial dosing, result in a low incidence of acute cholinergic effects, when compared to an equivalent dose in an immediate release formulation.

In embodiments, the disclosure includes sustained release formulations of donepezil or its salts, in matrix or reservoir forms.

In embodiments, the disclosure relates to sustained release formulations comprising donepezil or its salts and at least one release rate controlling material.

In embodiments, the disclosure relates to sustained-release formulations comprising donepezil or its salts and at least one release rate-controlling material, wherein a rate-controlling component is a hydrophilic, hydrophobic, enteric, water soluble or water-swellable material, or any combinations thereof.

In embodiments, the disclosure relates to sustained-release formulations comprising donepezil or its salts and a release rate-controlling material, wherein the rate-controlling material is a combination of enteric and water soluble or water swellable polymers, and wherein the donepezil-containing matrix does not include a water insoluble alkyl cellulose ingredient.

In embodiments, the disclosure includes pharmaceutical formulations comprising donepezil or its salts, wherein donepezil is in intimate contact with a release rate-controlling material.

In embodiments, the disclosure includes pharmaceutical formulations of donepezil in a waxy matrix core, optionally together with an enteric polymer, the core being coated with a hydrophilic material.

In embodiments, the disclosure includes release rate-controlling materials providing a pH dependent or pH independent release of donepezil or its salts from a formulation.

In embodiments, the disclosure includes dissolution, stability, and pharmacokinetic profiles of sustained release pharmaceutical formulations of donepezil comprising at least one release controlling material.

In embodiments, the disclosure includes methods of preparing pharmaceutical formulations of the present disclosure.

### DETAILED DESCRIPTION

Aspects of the present disclosure provide sustained release formulations comprising donepezil or its pharmaceutically acceptable salts and methods of preparing the sustained release formulations. Further aspects of the disclosure provide methods of using sustained release formulations to treat dementia of Alzheimer's disease.

The term “active agent” or “drug” is meant to include solvates (including hydrates) of the free compound or its salts, as well as various polymorphic crystalline and non-crystalline forms. Unless otherwise specified, the active agent is donepezil or a pharmaceutically acceptable salt thereof. For example, an active agent can include any optical isomers of the compound and any pharmaceutically acceptable salts thereof, either alone or in combination.

“Pharmaceutically acceptable” salts include derivatives of the disclosed compounds, wherein the parent compound is modified by forming acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines, alkali or organic addition salts of acidic residues such as carboxylic acids, and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric, and the like. Other acceptable inorganic salts include metal salts such as a sodium salt, potassium salt, cesium salt, and the like, and alkaline earth metal salts, such as a calcium salt, magnesium salt, and the like, and combinations comprising one or more of the foregoing salts. Pharmaceutically acceptable organic salts include: salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH, where n is 0-4, and the like; organic amine salts such as a triethylamine salt, pyridine salt, picoline salt, ethanolamine salt, triethanolamine salt, dicyclohexylamine salt, N,N′-dibenzylethylenediamine salt, and the like; amino acid salts such as arginate, asparginate, glutamate, and the like; and combinations comprising any of the foregoing salts.

The term “hydrophilic” for purposes of the present disclosure relates to excipients that are soluble and/or swellable in water, or have affinity toward water.

The term “enteric” for purposes of the present disclosure relates to excipients that do not dissolve or decompose to a significant extent in aqueous fluids having pH values about 4 or less, in an in vitro test, however, they will dissolve or decompose in aqueous fluids having pH values about 5 or greater, such as in the range of about 5 to 7, in the range of about 5 to 6, or in the range of about 5 to 5.5. In general, enteric polymers are intended to remain intact while in the highly acidic environment of the stomach, but not in higher pH environments such as the intestines.

The term “water soluble” for purposes of the present disclosure relates to excipients that dissolve to the extent required, in aqueous fluids having pH values in the range of about 1 to about 8, and is not particularly limited.

The term “water swellable” for purposes of the present disclosure relates to excipients that are relatively insoluble in water, but which can absorb at least two times their weight in water.

The term “pharmaceutical formulation” or “dosage form” for purposes of the present disclosure includes solid pharmaceutical products such as tablets, capsules, sachets, pills, or granules, which may be matrix based formulations, reservoir based formulations, multi-particulate based formulations, multi-layer formulations, resin formulations, osmotic formulations, gastro-retentive formulations, etc.

In embodiments, the disclosure relates to unit dosage forms that contain 1 mg to 60 mg, or 8 to 50 mg, or 8 to 36 mg, or 11 to 30 mg, of donepezil or a salt thereof.

A sustained-release dosage form is a form suitable for providing controlled release of the contained drug over an extended period of time (e.g., over 4 hours, 8 hours, 12 hours, 24 hours, etc.). Sustained-release dosage forms of donepezil may release the active agent at a rate independent of pH, for example, into fluids having pH values about 1 to about 8. Alternatively, sustained-release dosage forms may release the active agent at a rate dependent upon pH, for example, a lower rate of release at pH 1 and a higher rate of release at pH 7.5. In general, the sustained-release forms avoid “dose dumping” following oral administration. The sustained-release oral dosage forms can be formulated to provide for an increased duration of therapeutic action, allowing once-daily or less frequent dosing.

A sustained-release dosage form comprises a release rate-controlling material. The rate-controlling material can be associated with the formulation either in the form of a matrix or a coating. The rate-controlling material is a material that permits release of the active agent at a sustained rate into an aqueous medium.

In embodiments, the disclosure relates to sustained-release formulations comprising donepezil or its salts and at least one release rate-controlling material, wherein a rate-controlling component is hydrophilic, hydrophobic, enteric, or combinations thereof.

In embodiments, the disclosure relates to sustained-release formulations comprising donepezil or its salts and release rate-controlling material which is a combination of enteric and water soluble or water swellable polymers and optionally coated with enteric or water soluble or water swellable polymers.

In embodiments, the disclosure includes pharmaceutical formulations comprising donepezil or its salts, wherein donepezil is in intimate contact with a release rate-controlling material.

In embodiments, the disclosure includes pharmaceutical formulations of donepezil comprising a waxy matrix core optionally with enteric polymer, which is coated with hydrophilic material.

In embodiments, the disclosure includes release rate-controlling materials providing a pH dependent or pH independent release of donepezil or its salts from a formulation.

The term, “pH dependent” release rate-controlling materials for the purpose of this disclosure includes materials that permit release of the active agent into an aqueous medium depending upon the pH of the medium, such as enteric polymers.

The term, “pH independent” release rate-controlling materials for the purpose of this disclosure includes materials that affect the release of the active agent into aqueous media having different pH values.

Suitable pH independent release rate-controlling materials may include, for example, hydrophilic materials, hydrophobic materials, and combinations comprising one or more of the foregoing materials. The oral dosage forms can contain from about 1% to about 80% of rate-controlling material by weight of the formulation.

Suitable hydrophilic materials comprise water soluble or water swellable materials. Examples of such materials include hydroxyalkyl celluloses, hydroxyalkyl alkylcelluloses, and carboxyalkyl cellulose esters, for example, hydroxypropyl methylcelluloses (hypromelloses or HPMC), hydroxypropylcelluloses (HPC), and combinations comprising one or more of the foregoing materials. For the purposes of this disclosure, the release modifying agent may be present in a matrix, or in a coating covering the matrix. For the purposes of this disclosure, the concentration of hydrophilic material ranges from about 5% to about 50% by weight of the formulation.

In embodiments, pharmaceutical compositions comprise mixtures of water soluble materials of different viscosity grades, such as hydroxypropyl methylcelluloses and hydroxypropylcelluloses. These water soluble materials may be characterized by their viscosities in a 2% w/w aqueous solution as low viscosity (less than about 1 Pa·s, or less than about 1,000 cP), medium viscosity (about 1 Pa·s to about 10 Pa·s, or about 1,000 cP to about 10,000 cP), and high viscosity (greater than about 10 Pa·s, or greater than about 10,000 cP).

Hydroxypropyl methylcellulose polymers that are hydrophilic in nature and may be used in the present disclosure are sold in different viscosity grades such as those sold under the brand name Methocel™ available from Dow Chemical Co. Examples of hydroxypropyl methylcellulose polymers of a low viscosity grade include those available under the brand names Methocel E5, Methocel E-15 LV, Methocel E50 LV, Methocel K100 LV and Methocel F50 LV whose 2% by weight aqueous solutions have viscosities of 5 cP, 15 cP, 50 cP, 100 cP, and 50 cP, respectively. Examples of hydroxypropyl methylcellulose polymers having medium viscosity include those available under the brand names Methocel E4M and Methocel K4M, both of whose 2% by weight aqueous solutions have a viscosity of 4000 cP. Examples of hydroxypropyl methylcellulose polymers having high viscosity include those available under the brand names Methocel K15M and Methocel K100M whose 2% by weight aqueous solutions have viscosities of 15,000 cP and 100,000 cP, respectively. The hydroxypropyl methylcellulose polymers may be present in the pharmaceutical compositions of the present disclosure in amounts from about 0.1% to 50% by weight.

The hydroxypropylcellulose polymers that may be used in the present disclosure also include, for example, polymers available under the brand name Klucel™, available from Nippon Soda Co. Hydroxypropylcellulose polymers available under the brand names Klucel EF, Klucel LF, Klucel JF and Klucel GF, whose 2% by weight aqueous solutions have viscosities less than 1000 cP, are examples of low viscosity hydrophilic polymers. A hydroxypropylcellulose polymer available under the brand name Klucel ME whose 2% by weight aqueous solution has a viscosity in the range from 4,000-6,500 cP is a medium viscosity hydrophilic polymer. Hydroxypropyl cellulose polymers available sold as HPC-SL, HPC-L, and HPC-M, whose 2% by weight aqueous solutions have viscosities of 3-6 cP, 6-10 cP, and 150-400 cP, respectively, are examples of low viscosity hydrophilic polymers, while HPC-H has a viscosity of 1,000-4000 cP and is an example of a medium viscosity hydrophilic polymer. The hydroxypropylcellulose polymers may be present in an amount from about 0.1% to 50% by weight.

Water swellable substances suitable for making sustained release dosage forms are compounds that are able to expand when they are exposed to aqueous fluids, such as gastro-intestinal fluids. One or more water swellable substances may be present in a matrix or coating together with the active agent and optionally one or more pharmaceutically acceptable excipients.

Suitable substances which can be used as water swellable substances include, for example, low-substituted hydroxypropyl celluloses, e.g. L-HPC, cross-linked polyvinylpyrrolidones, e.g., PVP-XL, Kollidone™ CL and Polyplasdone™ XL, sodium carboxymethylcellulose, cross-linked sodium carboxymethylcellulose, e.g., Ac-di-sol™ and Primellose™, sodium starch glycolate, e.g., Primojel™ sodium carboxymethylcelluloses, e.g., Nymcel™ ZSB10, sodium carboxymethyl starches, e.g., Explotab™, ion-exchange resins, e.g., Dowex™ or Amberlite™ products, microcrystalline cellulose, e.g., Avicel™ products, starches and pregelatinized starches, e.g., Starch 1500™ and Sepistab ST200™, formalin-casein, e.g., Plas-Vita™, and combinations comprising one or more of the foregoing water swellable substances.

In embodiments, hydrophilic materials include polyalkylene oxides, polysaccharide gums, and crosslinked polyacrylic acids.

Suitable polyalkylene oxides, such as linear polymers of unsubstituted ethylene oxide, include Polyox™ products from The Dow Chemical Company, U.S., having molecular weights about 100,000-7,000,000. For example, poly(ethylene oxide)polymers having molecular weights about 4,000,000 and higher, such as about 4,500,000 to about 10,000,000, or about 5,000,000 to about 8,000,000, can be used. Other useful polyalkylene oxide polymers are made from propylene oxide, or mixtures of ethylene oxide and propylene oxide.

Polysaccharide gums, both natural and modified (semi-synthetic), can be used. Examples are dextran, xanthan gum, gellan gum, welan gum and rhamsan gum.

Crosslinked polyacrylic acids that can be used include those having properties similar to those described above for alkyl-substituted cellulose and polyalkylene oxide polymers. Useful crosslinked polyacrylic acids include those with viscosities about 4,000 to about 40,000 cP (for a 1% aqueous solution at 25° C.). Three specific examples are CARBOPOL™ grades 971P, 974P, and 934P (sold by The Lubrizol Corporation, Cleveland, Ohio, USA). Further examples are polymers known as WATER LOCK™, which are starch/acrylate/acrylamide copolymers available from Grain Processing Corporation, Muscatine, Iowa, USA.

The hydrophilicity and water swellability of these polymers cause the active agent-containing matrices to swell in size after oral administration, due to ingress of water. The release rate of an active agent from the matrix is primarily dependent upon the rate of water imbibition and the rate at which the active agent dissolves and diffuses from the swollen polymer, which in turn is related to the solubility and dissolution rate of the active agent, the active agent particle size, and the active agent concentration in the matrix.

Suitable “hydrophobic” materials are water-insoluble neutral or synthetic waxes, fatty alcohols such as lauryl, myristyl, stearyl, cetyl, or cetostearyl alcohol, fatty acids and derivatives thereof, including fatty acid esters such as such as glyceryl monostearate, glycerol monooleate, acetylated monoglycerides, stearin, palmitin, laurin, myristin, cetyl esters wax, glyceryl palmitostearate, glyceryl behenate, hydrogenated castor oils, cottonseed oils, fatty acid glycerides (mono-, di-, and tri-glycerides), hydrogenated fats, hydrocarbons, normal waxes, stearic acid, stearyl alcohol, materials having hydrocarbon backbones, and combinations comprising one or more of the foregoing materials. Suitable waxes include, but are not limited to, beeswax, Glycowax® (a N,N′-distearoylethyelenediamine, from Lonza), castor wax, carnauba wax, and wax-like substances.

A wax formulation is a solid dosage form comprising donepezil or a pharmaceutically acceptable salt thereof in a waxy matrix. The waxy matrix may be prepared by known tableting technologies such as wet granulation, dry granulation, or direct compression. Alternatively, a waxy matrix may be prepared by melting a suitable wax material and using the melt to granulate the active agent, optionally in combination with one or more other excipient materials. The matrix material comprises the waxy material and the active agent.

The wax material can be, for example, an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those having skill in the art. A typical cetyl esters wax, for example, has a molecular weight of about 470 to about 490 and is a mixture containing primarily esters of saturated fatty alcohols and saturated fatty acids.

The wax material can comprise a vegetable wax such as carnauba wax, a hydrogenated castor oil, glyceryl behenates, and combinations comprising one or more of the foregoing waxes. Hydrogenated castor oil is a hard wax with a high melting point, about 83-88° C. Hydrogenated castor oil is obtained by hydrogenation of virgin castor oil. It is mainly the triglyceride of 12-hydroxystearic acid.

When the waxy material is a hydrogenated castor oil and no other waxy material is used, the matrix can be coated with a functional coating. When the waxy material includes glyceryl behenates or carnauba wax, the matrix can be used without a coating, but may have either a cosmetic coating or a functional coating depending on the precise release profile and appearance desired. Sometimes combinations of waxes such as carnauba wax and glyceryl behenate, carnauba wax and castor wax, etc., may be used.

In embodiments, formulations include a rate-controlling material that is an “enteric polymer,” being insoluble in highly acidic environments such as the stomach, but being dissolved or decomposed in higher pH environments such as the intestines. Examples include polyvinylacetate phthalates (PVAP), alginic acid and its derivatives, hydroxypropyl methylcellulose acetate succinates (HPMCAS), cellulose acetate phthalates (CAP), methacrylic acid copolymers, hydroxypropyl methylcellulose succinates, cellulose acetate succinates, cellulose acetate hexahydrophthalates, hydroxypropyl methylcellulose hexahydrophthalates, hydroxypropyl methylcellulose phthalates (HPMCP), cellulose propionate phthalates, cellulose acetate maleates, cellulose acetate trimellitates, cellulose acetate butyrates, cellulose acetate propionates, methacrylic acid/methacrylate polymers (e.g., acid number 300 to 330 and also known as EUDRAGIT™ L from Evonik Industries, Germany, which is an anionic copolymer based on methacrylate, available as a powder, and also known as methacrylic acid copolymer, type A NF), methacrylic acid-methyl methacrylate copolymers, ethyl methacrylate-methylmethacrylate-chlorotrimethylammonium ethyl methacrylate copolymers, and the like, and combinations comprising one or more of the foregoing enteric polymers. Other examples include natural resins, such as shellac, sandarac resin, copal collophorium, and combinations comprising one or more of the foregoing polymers. Further examples of enteric polymers include synthetic resins bearing carboxyl groups. The methacrylic acid-acrylic acid ethyl ester 1:1 copolymer solid substance of the acrylic dispersion sold as EUDRAGIT L-100-55 is suitable.

Polymethacrylate enteric polymers are synthetic cationic and anionic polymers of dimethylaminoethyl methacrylates, methacrylic acid and methacrylic acid esters in varying molar ratios. Several different types are commercially available and may be purchased as dry powders, or in aqueous mixtures.

The pH independent release rate controlling polymers may include enteric acrylic polymers, for example, acrylic acid and methacrylic acid copolymers, methyl methacrylate copolymers, ethoxyethyl methacrylates, cyanoethyl methacrylates, aminoalkyl methacrylate copolymers, poly(acrylic acid), poly(methacrylic acid), methacrylic acid alkylamide copolymers, poly(methyl methacrylate), poly(methacrylic acid anhydride), methyl methacrylates, polymethacrylates, poly(methyl methacrylate)copolymers, polyacrylamides, aminoalkyl methacrylate copolymers, glycidyl methacrylate copolymers, and combinations comprising one or more of the foregoing polymers.

EUDRAGIT polymers are products of Evonik Industries AG, Essen, Germany. Commercially available Eudragit products include, but are not limited to, EUDRAGIT RL, EUDRAGIT RS, EUDRAGIT RL PO, EUDRAGIT RS PO, EUDRAGIT RD, EUDRAGIT L, EUDRAGIT S, EUDRAGIT L 100-5, EUDRAGIT NE 30D, and EUDRAGIT E 100. GANTREZ™ polymers are products of International Specialty Products, Parsippany, N.J. USA.

The polymers sold as EUDRAGIT have the following general repeating unit:

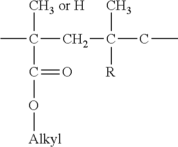

where R is COOH for the EUDRAGIT L products, R is COOCH₂N(CH₃)₂ for the EUDRAGIT E products, R is COOCH₃ for the EUDRAGIT NE 30 D product, and R is COOCH₂CH₂N⁺(CH₃)₃Cl⁻ for the EUDRAGIT E and EUDRAGIT RS products. The alkyl groups vary between different products, and have 1-4 carbons.

The United States Pharmacopoeia and National Formulary describes “methacrylic acid copolymer” as a fully polymerized copolymer of methacrylic acid and an acrylic or methacrylic ester. Three types of copolymers, namely Type A, Type B, and Type C, are defined in the monograph. They vary in their methacrylic acid content and solution viscosity. Type C may contain suitable surface-active agents. Two additional polymers, Type A (e.g., EUDRAGIT RL) and Type B (e.g., EUDRAGIT RS), are referred to as “ammonio methacrylate copolymers,” consisting of fully polymerized copolymers of acrylic and methacrylic acid esters with a low content of quaternary ammonium groups, are also described.

Other enteric materials include alginic acid derivatives such as, sodium alginate. Sodium alginate consists chiefly of the sodium salt of alginic acid, which is a mixture of polyuronic acids composed of residues of D-mannuronic acid and L-guluronic acid.

In embodiments, the disclosure includes sustained-release formulations of donepezil or its salts, wherein a formulation is a matrix or reservoir type.

In embodiments, the disclosure relates to sustained-release formulations of donepezil or its salts wherein a formulation is a monolithic or multi-particulate system.

In embodiments, the disclosure includes pharmaceutical formulations comprising donepezil or its salts, wherein donepezil is in intimate contact with a rate-controlling material, such as a co-precipitate having the characteristics of a solid solution.

In embodiments, the disclosure relates to sustained-release formulations comprising donepezil or its salts and a release rate-controlling material such as a combination of enteric polymer and water soluble or water swellable polymer, optionally coated with a water soluble or water swellable polymer.

The active agent in sustained-release form can include a plurality of particulate substrates comprising donepezil, which substrates are coated with a sustained-release coating comprising a rate-controlling material. The sustained-release preparations may thus be made in conjunction with a multi-particulate system, such as beads, ion-exchange resin beads, spheroids, microspheres, seeds, pellets, granules, and other multi-particulate systems, in order to obtain a desired release profile of the active agent. A multi-particulate system can be presented in capsules or alternatively may be compressed to form tablets.

In addition, the profile of donepezil release from the formulations can be altered, for example, by using more than one rate controlling material, varying the thickness of the rate controlling material, changing the particular rate controlling material used, altering the relative amounts of rate controlling material, altering the manner in which the plasticizer is added (e.g., when the sustained-release coating is derived from an aqueous dispersion of hydrophobic material), by varying the amount of plasticizer relative to rate controlling material, by the inclusion of additional ingredients or excipients, by altering the method of manufacture, etc.

When the release rate controlling material is present in a coating, additional excipients may be incorporated for a desired release profile. For the purpose of the present disclosure, these excipients are termed “release-modifying agents,” which affect the release properties of a rate-controlling material. The release-modifying agents may, for example, function as pore formers. Pore formers can be organic or inorganic, and include materials that can be dissolved, extracted or leached from the coating, in the environment of use. A pore former can comprise one or more hydrophilic materials, such as hydroxypropyl methylcelluloses, hydroxypropyl celluloses, polycarbonates comprising linear polyesters of carbonic acid in which carbonate groups recur in the polymer chain, and combinations comprising one or more of the foregoing release-modifying agents. Alternatively, a pore former may be a small molecule such as lactose, a metal stearate, and combinations comprising one or more of the foregoing release-modifying agents.

Delayed-release formulations may comprise a core, a first coating, and optionally a second coating. The core may include donepezil and excipients, such as a lubricant, a binder and/or filler, and optionally a glidant, as well as other excipients.

The coatings may contain a water-insoluble polymer and/or a water-soluble polymer, and a plasticizer. The relative proportions of ingredients, notably the ratios of water-insoluble, film-forming polymer to water-soluble polymer, can be varied depending on the release profile to be obtained. The coating may comprise an enteric polymer of the methacrylic type, and optionally a plasticizer.

In embodiments, the disclosure includes formulations that may have substantially zero order, first order, and second order release rate profiles. The rate of diffusion of the active agent out of a matrix can be slowed by increasing the active agent particle size, by the choice of polymer used in the matrix, and/or by the choice of molecular weight of the polymer. The matrix is a relatively high molecular weight polymer that swells upon ingestion, and that promotes gastric retention during the fed mode. The rate-limiting factor in the release of the active agent may be therefore controlled diffusion of the active agent from the matrix rather than erosion, dissolving, or chemical decomposition of the matrix.

For highly soluble active agents such as donepezil or its salts, the swelling of the polymeric matrix can achieve two objectives: (i) the tablet swells to a size large enough to cause it to be retained in the stomach during the fed state; and (ii) it retards the rate of diffusion of the highly soluble active agent long enough to provide multi-hour, controlled delivery of the active agent into the stomach.

The amount of rate controlling material relative to the active agent can vary, depending on the active agent release rate desired and on the polymer, its molecular weight, and excipients that may be present in the formulation.

Apart from the above discussed functional excipients such as hydrophobic materials and lipophilic materials, water swellable materials, and hydrophilic materials, the dosage forms can further comprise various other pharmaceutically acceptable excipients such as any one or more of diluents, binders, glidants, lubricants, colouring agents, and coating materials.

Useful diluents include, but are not limited to, starches, lactose, mannitol, Pearlitol™ SD 200, celluloses, confectioner's sugar, and the like. Different grades of lactose include, but are not limited to, lactose monohydrate, lactose DT (direct tabletting), lactose anhydrous, Flowlac™ (available from Meggle Products), Pharmatose™ (available from DMV), and others. Different grades of starches include, but are not limited to, maize starch, potato starch, rice starch, wheat starch, pregelatinized starches (commercially available as PCS PC10 from Signet Chemical Corporation) and Starch 1500, Starch 1500 LM grade (low moisture content grade) from Colorcon, fully pregelatinized starches (commercially available as National 78-1551 from Essex Grain Products) and others. Different celluloses that can be used include crystalline celluloses and powdered celluloses. Examples of crystalline cellulose products include but are not limited to CEOLUS™ KG801, Avicel™ PH101, PH102, PH301, PH302 and PH-F20, microcrystalline cellulose 114, and microcrystalline cellulose 112. Other useful diluents include, but are not limited to, carmellose, sugar alcohols such as mannitol, sorbitol and xylitol, calcium carbonate, magnesium carbonate, dibasic calcium phosphate, directly compressible grades of dibasic calcium phosphate (Emcompress™), and tribasic calcium phosphate.

Useful binders include, but are not limited to, hydroxypropyl celluloses (Klucel™ LF), Nisso HPC-L (Nippon Soda Co. Ltd.), HPC-EXF, low viscosity hydroxypropyl methylcelluloses (HPMC or hypromelloses, e.g., Methocel™) polyvinylpyrrolidones or povidones (e.g., grades PVP-K25, PVP-K29, PVP-K30, PVP-K90, etc.), copovidones, (e.g., Plasdone™ S 630, a random copolymer of N-vinyl-2-pyrrolidone and vinyl acetate), powdered acacia, gelatin, guar gum, carbomers (e.g. Carbopol™ products), water-soluble methylcelluloses, polymethacrylates, and starches.

Solvents that are useful in processing include, but are not limited to, water, methanol, ethanol, isopropanol, butanols, acidified ethanol, acetone, diacetone, polyols, polyethers, oils, esters, alkyl ketones, methylene chloride, methyl acetate, ethyl acetate, isopropyl acetate, castor oil, ethylene glycol monoethyl ether, diethylene glycol monobutyl ether, diethylene glycol monoethyl ether, dimethylsulphoxide, dimethylformamide, tetrahydrofuran, and any mixtures thereof.

Glidants or anti-sticking agents can be used, including but not limited to talc, silica derivatives, colloidal silicon dioxide and the like or mixtures thereof, and lubricants that can be used include, but are not limited to, stearic acid and stearic acid derivatives such as magnesium stearate, calcium stearate, zinc stearate, sucrose esters of fatty acid, polyethylene glycol, talc, sodium stearyl fumarate, zinc stearate, castor oils, and waxes.

Various useful colourants include, but are not limited to, Food Yellow No. 5, Food Red No. 2, Food Blue No. 2, and the like, food lake colorants, and iron oxides.

If desired, an outer continuous phase in the form of a polymer film may be used, optionally containing additional adjuvants for coating processing such as plasticizers, polishing agents, colorants, pigments, antifoam agents, opacifiers, antisticking agents, and the like. Suitable polymers include the hydrophilic and hydrophobic polymers that have been described above.

Various plasticizers include, but are not limited to, castor oil, diacetylated monoglycerides, dibutyl sebacate, diethyl phthalate, glycerin, polyethylene glycols, propylene glycol, triacetin, triethyl citrate. Also, mixtures of plasticizers may be utilized. The type of plasticizer depends upon the type of coating agent. A plasticizer is frequently present in an amount ranging from 5% to 30, based on the total weight of the film coating.

An opacifier like titanium dioxide may also be present, in amounts ranging from about 10% to about 20%, based on the total weight of the coating. When colored tablets are desired then the colour is normally applied in the coating. Consequently, colouring agents and pigments may be present in the film coating. Various colouring agents include but not limited to iron oxides, which can be red, yellow, black or blends thereof.

Anti-adhesives are frequently used in the film coating process to avoid sticking effects during film formation and drying. An example of an anti-adhesive for this purpose is talc. The anti-adhesive typically is present in the film coating in an amount of about 5% to 15%, based upon the total weight of the coating.

As alternatives for the above coating ingredients, sometimes pre-formulated coating products such as those sold as OPADRY™ (supplied by Colorcon) will be used, for example Opadry Blue 13B50579 or Opadry White OY 59800. The products sold in a solid form require only mixing with a liquid before use.

The dosage forms may include additives that impart some degree of hydrophobic character, to further retard the release rate of the active agent into a gastric fluid. One example of such a hydrophobic rate controlling material is glyceryl monostearate. Other examples are fatty acids and salts of fatty acids such as sodium myristate.

A tablet matrix should not include any alkyl cellulose ingredient, such as ethylcellulose, that is not water soluble, but alkyl celluloses generally can be present in tablet coatings, if desired.

The pharmaceutical formulations of the present disclosure may be prepared using any of techniques such as dry granulation, wet granulation, direct compression, melt granulation, extrusion-spheronization, etc., including combinations thereof.

Equipment suitable for processing pharmaceutical formulations of the present disclosure include any one, or a combination of, mechanical sifters, blenders, roller compactors, granulators (rapid mixer or fluid bed granulator), fluid bed dryers, compression machines, rotating bowls or coating pans, etc.

Aspects of the present disclosure further relate to processes for manufacturing pharmaceutical formulations of the present disclosure, wherein embodiments comprise:

1) Sifting drug substance, hydrophobic or lipophilic materials, and other excipients such as diluents, disintegrants, etc., through a sieve and mixing.

2) Granulating step 1) materials using a solvent or granulating fluid.

3) Drying the granules.

4) Sifting the dried granules and extragranular excipients through a sieve.

5) Blending sifted granules and extragranular excipients and adding a lubricant to the blend.

6) Compressing the final lubricated blend into tablets or filling into capsules.

7) Coating tablets or capsules with hydrophobic, lipophilic, or hydrophilic materials, together with coating adjuvants.

Alternatively, step 1) materials can be blended with extragranular excipients and lubricants and compressed into tablets, or filled into capsules, optionally followed by applying a sustained release coating. As another alternative, step 1) materials may be compacted and milled through a screen, to form granules that can be blended with extragranular materials and lubricants and compressed into tablets, or filled into capsules.

Alternatively, when the excipients are wax based, then excipients including a wax are melted and drug dispersed in the melt, followed by adsorbing onto inert materials or granulating using mixtures of excipients, to form pellets or granules which may be blended with other excipients and compressed into tablets or filled into capsules.

Tablets or capsules prepared as above can be subjected to in vitro drug dissolution evaluations according to Test 711 “Dissolution” in United States Pharmacopoeia 29, United States Pharmacopeial Convention, Inc., Rockville, Md., 2005 (“USP”) to determine the rate at which the active substance is released from the dosage forms, and content of active ingredient can conveniently be determined in solutions using techniques such as high performance liquid chromatography.

In embodiments, the disclosure includes the use of packaging materials such as containers and closures of high-density polyethylene (HDPE), low-density polyethylene (LDPE) and/or polypropylene and/or glass, and blisters or strips composed of aluminum, high-density polypropylene, polyvinylchloride, polyvinylidine dichloride, etc.

In addition to the embodiments where donepezil is the only active agent, the disclosure includes combination dosage forms that also contain other active agents useful in the treatment of conditions such as Alzheimer's dementia, for the improvement of cognition. Such combinations are useful for treating both the psychosis and memory deficits of Alzheimer's dementia. Suitable cognition enhancers for use as a combination therapy with donepezil includes, for example, memantine, metrifonate, rivastigmine, tacrine, galantamine, or a combination comprising at least one of the foregoing cognition enhancers.

The invention further includes combinations that may contain any of an antidepressant, an antipsychotic, and the active metabolite of donepezil, 6-O-desmethyldonepezil, as an additional active agent. Suitable antidepressants for use in the combination include, for example, citalopram, citalopram HBr, fluvoxamine, paroxetine, fluoxetine, sertraline, amitriptyline, desipramine, nortriptyline, venlafaxine, phenelzine, tranylcypromine, mirtazepine, nefazodone, trazodone, bupropion, or a combination comprising at least one of the foregoing antidepressants. Exemplary antipsychotics include clozapine, risperidone, olanzapine, quetiapine, loxapine, ziprasidone, or a combination comprising at least one of the foregoing antipsychotics.

The invention further includes combinations with other drugs such as, aluminum hydroxide, magnesium hydroxide, aluminum carbonate, calcium carbonate, and sodium bicarbonate; histamine-2 antagonists (H2-antagonists), examples of which include cimetidine, famotidine, nizatidine, ranitidine; and proton pump inhibitors, such as lansoprazole, omeprazole, pantoprazole, esomeprazole, esomeprazole magnesium, and rabeprazole.

The following examples further describe certain specific aspects and embodiments of the disclosure and demonstrate the practice and advantages thereof. It is to be understood that the examples are provided only for purposes of illustration and are not intended to limit the scope of the disclosure in any manner.

### EXAMPLE 1

### Sustained Release Donepezil Formulation

| Ingredient | mg/Tablet |
|---|---|
| Donepezil hydrochloride | 23 |
| Cetostearyl alcohol | 15 |
| Microcrystalline cellulose PH102 | 33.25 |
| Microcrystalline cellulose PH102 | 55 |
| EUDRAGIT ™ RL* | 12 |
| EUDRAGIT RS** | 8 |
| Talc | 1.5 |
| Magnesium stearate | 0.75 |
| Colloidal silicon dioxide | 1.5 |

*EUDRAGIT RL chemically is poly(ethylacrylate, methylmethacrylate, trimethyl ammonioethyl methacrylate chloride) 1:2:0.2, supplied by Evonic Industries, Germany.
**EUDRAGIT RS chemically is poly(ethylacrylate, methylmethacrylate, trimethyl ammonioethyl methacrylate chloride) 1:2:0.1, supplied by Evonic Industries, Germany.

Manufacturing Process:

1) Cetostearyl alcohol is melted at about 50° C.

2) Donepezil hydrochloride and microcrystalline cellulose PH102 (first quantity) are sifted through an ASTM #40 mesh sieve.

3) The mixture from step 2 is granulated using melted cetostearyl alcohol of step 1.

4) Granules are dried for 1 hour at 30° C. and the dried granules are sifted through an ASTM #30 mesh sieve.

5) Microcrystalline cellulose PH102 (second quantity), EUDRAGIT RL PO, and EUDRAGIT RS PO are sifted through an ASTM #40 mesh sieve.

6) Granules from step 4 and the mixture from step 5 are sifted together for about 10 minutes.

7) Talc, colloidal silicon dioxide, and magnesium stearate are sifted through an ASTM #60 mesh sieve.

8) Mixtures from steps 6 and step 7 are blended for about 5 minutes.

9) The final blend is compressed into tablets having 12 to 15 Kp hardness.

### EXAMPLE 2

### Sustained Release Donepezil Formulation

| Ingredient | mg/Tablet |
|---|---|
| Donepezil hydrochloride | 23 |
| Hydroxypropyl methylcellulose K100 M | 85 |
| EUDRAGIT RS | 45 |
| Pregelatinized starch | 24 |
| Anhydrous lactose | 20 |
| Colloidal silicon dioxide | 1.5 |
| Magnesium stearate | 1.5 |

Manufacturing Process:

1. Donepezil hydrochloride is mixed geometrically with HPMC K100 M, Eudragit RS, anhydrous lactose, pre-gelatinized starch, and colloidal silicon dioxide. The mixture is sifted three times through an ASTM #40 sieve.

2. Magnesium stearate is sifted through an ASTM #60 mesh sieve.

3. The mixture of step 1 is blended with magnesium stearate of step 2.

4. The lubricated blend from step 3 is compressed into tablets.

### EXAMPLES 3-5

### Sustained Release Donepezil Formulations

| Ingredient | Example 3 | Example 4 | Example 5 |
|---|---|---|---|
| Donepezil hydrochloride | 23 | 23 | 23 |
| Polyvinyl alcohol | 47 | 55 | — |
| Hydroxyethyl cellulose (HEC) | 40 | — | — |
| HPC HF | — | 40 | — |
| HPMC K100M | — | — | 47 |
| EUDRAGIT L 100-55 | — | — | 40 |
| Mannitol | 25 | — | 25 |
| Pregelatinized starch | | 30 | — |
| Anhydrous lactose | 20 | 25 | 20 |
| Colloidal silicon dioxide | 3 | 5 | 3 |
| Talc | 2 | 2 | 2 |

Manufacturing Process:

1. Donepezil hydrochloride is mixed geometrically with the required excipients (except talc) and the mixtures are sifted through an ASTM #40 mesh sieve.

2. Talc is sifted through an ASTM #60 mesh sieve.

3. The mixture of step 1 is blended with talc of step 2.

4. The lubricated blend from step 3 is compressed into tablets.

### EXAMPLE 6

### Sustained Release Donepezil Formulation

| Ingredient | mg/Tablet |
|---|---|
| Donepezil hydrochloride | 23 |
| Hydroxypropyl methylcellulose K 100M | 80 |
| EUDRAGIT RS PO | 20 |
| EUDRAGIT RSPO | 38 |
| Povidone K 30 | 15 |
| Powdered cellulose | 70 |
| Colloidal silicon dioxide | 2 |
| Magnesium stearate | 2 |
| Methanol* | q.s. |

*Evaporates during processing.

Manufacturing Process:

1. Donepezil hydrochloride, HPMC K 100 M and EUDRAGIT RLPO are sifted through an ASTM #40 mesh sieve.

2. Povidone is dissolved in methanol.

3. The mixture from step 1 is granulated with the solution from step 2.

4. The granules are passed through an ASTM #24 mesh sieve and dried at 50° C. for 30 minutes.

5. Powdered cellulose and colloidal silicon dioxide (previously passed through an ASTM #40 mesh sieve) are added to the dried granules.

6. The mixture from step 5 is blended with magnesium stearate and compressed into tablets.

### EXAMPLES 7-8

### Sustained Release Donepezil Formulation

(mg/Tablet)

| Ingredient | Example 7 | Example 8 |
|---|---|---|
| Donepezil hydrochloride | 23 | 23 |
| Eudragit L 100 55 | 40 | 65 |
| Hypromellose K 100 LV CR | 21 | 28 |
| Lactose monohydrate | 94 | 62 |
| Hydroxypropyl cellulose (HPC-L) | 5 | 5 |
| Hydroxypropyl cellulose (HPC-EXF) | 5 | 5 |
| Magnesium stearate | 2 | 2 |
| Opadry White 59800* | 8 | 8 |

*Opadry White 59800 is a formulated coating product from Colorcon, containing hypromellose 2910, polyethylene glycol 400, and titanium dioxide.

Manufacturing Process:

A) Sifting:

1. Lactose monohydrate is passed through an ASTM #20 mesh sieve.

2. Donepezil hydrochloride, Eudragit L 100 55, HPC EXF and HPMC K100 LV CR are sifted through an ASTM #20 mesh sieve and combined with step 1.

B) Granulation:

1. Hydroxypropyl cellulose is slowly added to water, with stirring to form a clear solution.

2. The mixture of A is granulated using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization and an inlet air temperature 50±10° C. for 30 minutes. Loss on drying (LOD) measured at 105° C. for 5 minutes is ≦3% w/w.

C) Milling:

1. Dried granules are milled through a Quadro co-mill fitted with 32R screen at 2500 rpm speed.

D) Blending and Lubrication:

1. Milled granules are blended for 5 minutes in a double cone blender.

2. Magnesium stearate is sifted through an ASTM #30 mesh sieve, added to the double cone blender and blended for 7 minutes.

E) Compression:

1. The blend is compressed into 8 mm round tablets having hardness 10-14 Kp, thickness 3.9-4.3 mm, and friability 0.01%.

F) Film Coating:

1. Opadry OY 58900 (white) is mixed with iron oxide red and iron oxide yellow and slowly added into water under continuous stirring. The stirring is continued for 45 minutes until a smooth homogeneous suspension is obtained.

2. Compressed tablets are coated with the Opadry dispersion in a coating pan, using a spray rate 5 g/minute, pan speed 8 rpm, inlet temperature 60° C., and a weight gain of 4%. Coated tablets are cured at 50° C. for 20 minutes.

### EXAMPLES 9-11

### Sustained Release Donepezil Formulations

| | mg/Tablet | | |
|---|---|---|---|
| Ingredient | Example 9 | Example 10 | Example 11 |
| Donepezil hydrochloride | 23 | 23 | 23 |
| Eudragit L 100 55 | 40 | 40 | 40 |
| Hypromellose K 100 LV | 43 | 28 | 35 |
| Lactose monohydrate | 72 | 87 | 80 |
| Hydroxypropyl cellulose | 5 | 5 | 5 |
| (HPC-L Nisso) | | | |
| Hydroxypropyl cellulose | 5 | 5 | 5 |
| (HPC-EXF) | | | |
| Magnesium stearate | 2 | 2 | 2 |
| Opadry White 59800 | 8.12 | 8.12 | 8.12 |
| Iron oxide yellow | 0.14 | 0.14 | 0.14 |
| Iron oxide red | 0.29 | 0.29 | 0.29 |

Manufacturing processes: similar to the process for Example 7, except that colorants iron oxide (red and yellow) are added in the film coating. The procedure for preparation of a film coating dispersion (with colorants) is described below:

Opadry White 58900 is mixed with iron oxide red and iron oxide yellow and slowly added to water, and the dispersion is stirred for 45 minutes. The dispersion is passed through a colloid mill and maintained under constant stirring until the completion of the coating procedure.

Samples of tablets prepared in Examples 9, 10, and 11, and the commercial product ARICEPT 23 mg tablets, are tested for their drug dissolution profiles and results are in Table 1. The following parameters are used:

Profile 1: Dissolution in 900 mL of 0.1 N HCl medium, using USP type 2 apparatus, stirred at 50 rpm for 14 hours.

Profile 2: Dissolution in 900 mL of pH 5.5 sodium phosphate buffer medium, using USP type 2 apparatus, stirred at 50 rpm for 14 hours.

Profile 3: Dissolution in 900 mL of 0.1 N HCl medium for 120 minutes, then in 900 mL of 6.8 pH sodium phosphate buffer medium, using USP type 2 apparatus, stirred at 25 rpm.

TABLE 1

| | Cumulative % of Drug Dissolved | | | |
|---|---|---|---|---|
| Hours | Example 9 | Example 10 | Example 11 | ARICEPT |

Profile 1

| | | | | |
|---|---|---|---|---|
| 1 | 30 | 35 | 33 | 30 |
| 2 | 49 | 56 | 52 | 45 |
| 3 | 63 | 70 | 67 | 54 |
| 4 | 73 | 81 | 78 | 62 |
| 5 | 82 | 88 | 86 | 69 |
| 6 | 88 | 93 | 91 | 75 |
| 8 | 95 | 98 | 96 | 83 |
| 10 | 99 | 100 | 99 | 88 |
| 12 | 100 | 101 | 100 | 92 |
| 14 | 102 | 102 | 101 | 95 |

Profile 2

| | | | | |
|---|---|---|---|---|
| 1 | 24 | 31 | 31 | 31 |
| 2 | 37 | 46 | 48 | 43 |
| 3 | 48 | 57 | 61 | 52 |
| 4 | 57 | 65 | 71 | 59 |
| 5 | 63 | 70 | 78 | 66 |
| 6 | 69 | 76 | 83 | 72 |
| 8 | 78 | 81 | 90 | 78 |
| 10 | 82 | 83 | — | 80 |
| 12 | 84 | 84 | 92 | 81 |
| 14 | 85 | 85 | 92 | 83 |

Profile 3

| | | | | |
|---|---|---|---|---|
| 1 | 25 | 31 | 27 | 28 |
| 2 | 41 | 48 | 45 | 43 |
| 3 | 51 | 60 | 55 | 52 |
| 4 | 58 | 67 | 62 | 62 |
| 5 | 65 | 74 | 69 | 70 |
| 6 | 71 | 81 | 75 | 78 |
| 8 | 80 | 89 | 84 | 90 |
| 10 | 86 | 94 | 90 | 96 |
| 12 | 91 | 97 | 93 | 97 |
| 14 | 94 | 98 | 95 | 98 |

Thirty tablets prepared in Examples 9 and 10 are packaged in closed HDPE containers, together with a 1 g silica gel desiccant pouch, and stored at 40° C. and 75% relative humidity or 25° C. and 60% RH conditions for three months. Impurity content and dissolution testing results of initial and stored samples are shown in Table 2. The dissolution study is conducted in 900 mL of 0.1N HCl media, using USP type 2 apparatus, stirred at 50 rpm for 14 hours. Drug assay and drug-related impurity values are expressed as percentages of the label donepezil content.

TABLE 2

| | | 40° C. and 75% RH | | | 25° C. and |
|---|---|---|---|---|---|
| | | 1 | 2 | 3 | 60% RH |
| Parameter | Initial | Month | Months | Months | 3 Months |

Example 9

| Drug assay | | 98.8 | 100.7 | 100.7 | 98.5 | 100 |
|---|---|---|---|---|---|---|
| DON-2* impurity | | 0.011 | 0.012 | 0.012 | 0.013 | 0.012 |
| N-oxide** impurity | | 0.023 | 0.024 | 0.026 | 0.028 | 0.027 |
| Highest unidentified | | 0.027 | 0.043 | 0.042 | 0.056 | 0.068 |
| impurity | | | | | | |
| Total impurities | | 0.12 | 0.12 | 0.11 | 0.15 | 0.16 |
| Cumulative | 1 hr. | 29 | 30 | 30 | 29 | 28 |
| donepezil | 3 hrs. | 61 | 63 | 62 | 61 | 61 |
| release (%) | 12 hrs. | 102 | 100 | 99 | 100 | 101 |

Example 10

| Drug assay | | 98.9 | 100.8 | 99.9 | 99.8 | 99.6 |
|---|---|---|---|---|---|---|
| DON-2* impurity | | 0.009 | 0.011 | 0.012 | 0.014 | 0.012 |
| N-oxide** impurity | | 0.018 | 0.027 | 0.029 | 0.026 | 0.027 |
| Highest unidentified | | 0.027 | 0.044 | 0.044 | 0.069 | 0.062 |
| impurity | | | | | | |
| Total impurities | | 0.12 | 0.13 | 0.11 | 0.19 | 0.16 |
| Cumulative | 1 hr. | 34 | 34 | 34 | 37 | 38 |
| donepezil | 3 hrs. | 69 | 69 | 67 | 75 | 73 |
| release (%) | 12 hrs. | 102 | 100 | 100 | 100 | 100 |

*DON-2 is 5,6-Dimethoxy-2-(4-pyridyl)methylindan-1-one.
**N-Oxide is the donepezil N-oxide impurity.

### EXAMPLES 12-15

### Sustained Release Donepezil Formulations

| | mg/Tablet | | | |
|---|---|---|---|---|
| | Example | Example | Example | Example |
| Ingredient | 12 | 13 | 14 | 15 |

Core

| | | | | |
|---|---|---|---|---|
| Donepezil hydrochloride | 23 | 23 | 23 | 23 |
| Eudragit L 100 55 | 40 | 40 | — | — |
| Hypromellose K 100 LV | 28 | 28 | 57 | 45 |
| Lactose monohydrate | 87 | 87 | 98 | 110 |
| Hydroxypropyl cellulose | 5 | 5 | 5 | 5 |
| (HPC-L Nisso) | | | | |
| Hydroxypropyl cellulose | 5 | 5 | 5 | 5 |
| (HPC-EXF) | | | | |
| Magnesium stearate | 2 | 2 | 2 | 2 |

Sustained Release Coating

| | | | | |
|---|---|---|---|---|
| Eudragit L 30 D 55 | 5.3 | 8.67 | 2.65 | 8.67 |
| Sodium hydroxide | 0.02 | 0.02 | 0.01 | 0.02 |
| Hypromellose 3 cps | 21.2 | 26 | 10.6 | 26 |
| Triethyl citrate | 0.57 | 0.95 | 0.285 | 0.95 |
| Talc | 1.425 | 2.375 | 0.7125 | 2.375 |

Manufacturing Process:

A) Sifting:

1. Lactose monohydrate is passed through an ASTM #20 mesh sieve.

2. Donepezil hydrochloride, Eudragit L 100 55, HPC EXF, and HPMC K100 LV CR are passed through an ASTM #20 mesh sieve and combined with step 1.

B) Granulation:

1. Hydroxypropyl cellulose is slowly added to water, with stirring to form a clear solution.

2. The mixture of A is granulated using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization and an inlet air temperature 50±10° C. for 30 minutes. LOD measured at 105° C. for 5 minutes is ≦3% w/w.

C) Milling:

1. Dried granules are milled through a Quadro co-mill fitted with 32R screen at 2500 rpm speed.

D) Blending and Lubrication:

1. Milled granules are blended for 5 minutes in a double cone blender.

2. Magnesium stearate is sifted through an ASTM #30 mesh sieve, added to the double cone blender and blended for 7 minutes.

E) Compression:

1. The blend is compressed into 8 mm round tablets having hardness 10 14 Kp, thickness 3.9-4.3 mm, and friability 0.01%.

F) Sustained Release Coating:

1. HPMC 3 cps is added to water and stirred to form a clear solution.

2. Talc is added to water and homogenized for 10 minutes.

3. Eudragit L30 D 55 is added to water and neutralized with sodium hydroxide solution to pH 5.

4. The materials of steps 1-3 are combined and triethyl citrate is added to the mixture with continuous stirring for 30 minutes.

5. Compressed tablets are coated with the dispersion of step 4 in a coating pan, using a spray rate 5 g/minute, pan speed 8 rpm, inlet temperature 60° C., and a weight gain of 4%. Coated tablets are cured at 50° C. for 20 minutes.

Samples of the tablets prepared in Examples 12-15 are tested for their drug dissolution profiles, and results are shown in Table 3. Dissolution is conducted in 900 mL of 0.1N HCl media, using USP type 2 apparatus, stirred at 50 rpm for 14 hours.

TABLE 3

| | Cumulative % of Drug Dissolved | | | |
|---|---|---|---|---|
| Hours | Example 12 | Example 13 | Example 14 | Example 15 |
| 1 | 32 | 27 | 28 | 23 |
| 2 | 53 | 50 | 47 | 44 |
| 3 | 68 | 67 | 65 | 61 |
| 4 | 78 | 80 | 78 | 74 |
| 5 | 87 | 89 | 88 | 84 |
| 6 | 92 | 94 | 96 | 92 |
| 8 | 96 | 98 | 103 | 97 |
| 10 | 97 | 100 | 104 | 97 |
| 12 | 98 | 101 | 104 | 97 |
| 14 | 99 | 102 | 105 | 98 |

### EXAMPLES 16-18

### Sustained Release Donepezil Formulations

| | mg/Tablet | | |
|---|---|---|---|
| Ingredient | Example 16 | Example 17 | Example 18 |
| Donepezil HCl | 23 | 23 | 23 |
| Eudragit L 100 55 | 40 | 40 | 40 |
| HPMC K100LV | 32 | — | |
| Hydrogenated castor oil | — | 40 | 25 |
| Lactose monohydrate | 72 | 75 | 90 |
| Hydroxypropyl cellulose | 5 | 5 | 5 |
| (HPC-L) | | | |
| Hydrogenated castor oil | 11 | — | — |
| Hydroxypropyl cellulose | 5 | 5 | 5 |
| (HPC-EXF) | | | |
| Magnesium stearate | 2 | 2 | 2 |
| Opadry White 58900 | 8.12 | 8.12 | 8.12 |
| Iron oxide red | 0.29 | 0.29 | 0.29 |
| Iron oxide yellow | 0.14 | 0.14 | 0.14 |

Manufacturing Process:

A) Sifting:

1. Lactose monohydrate is passed through an ASTM #20 mesh sieve.

2. Donepezil hydrochloride, Eudragit L 100 55, and HPMC K100 LV CR or hydrogenated castor oil, are sifted through an ASTM #20 mesh sieve and combined with step 1.

B) Granulation:

1. Hydroxypropyl cellulose is slowly added to water, with stirring to form a clear solution.

2. The mixture of A is granulated using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization at an inlet air temperature 50±10° C. for 30 minutes. LOD measured at 105° C. for 5 minutes is ≦3% w/w.

C) Milling:

1. Dried granules are milled through a Quadro co-mill fitted with 32R screen at 2500 rpm speed.

D) Blending and Lubrication:

1. Milled granules are blended for 5 minutes in a double cone blender.

2. Magnesium stearate and HPC EXF are sifted through ASTM #30 mesh sieve, added to the double cone blender and blended for 7 minutes.

E) Compression:

1. The blend is compressed into 8 mm round tablets having hardness 10-14 Kp, thickness 3.9-4.3 mm, and friability 0.01%.

F) Film Coating:

1. Opadry White 58900 is mixed with iron oxide red and iron oxide yellow and slowly added to water, and the dispersion is stirred for 45 minutes.

2. Compressed tablets are coated with the Opadry dispersion in a coating pan, using a spray rate 5 g/minute, pan speed 8 rpm, inlet temperature 60° C., and a weight gain of 4%. Coated tablets are cured at 50° C. for 20 minutes.

Samples of the tablets prepared as per example 17-19 are tested for their drug dissolution profiles listed in Table 4. Dissolution was conducted in 900 mL of 0.1N HCl media, using USP type 2 apparatus, stirred at 50 rpm for 14 hours.

TABLE 4

| | Cumulative % of Drug Dissolved | | |
|---|---|---|---|
| Hours | Example 16 | Example 17 | Example 18 |
| 1 | 31 | 32 | 37 |
| 2 | 46 | 42 | 50 |
| 3 | 58 | 49 | 59 |
| 4 | 68 | 55 | 66 |
| 5 | 76 | 60 | 71 |
| 6 | 83 | 62 | 75 |
| 8 | 92 | 70 | 82 |
| 10 | 96 | 76 | 86 |
| 12 | 98 | 79 | 89 |
| 14 | 100 | 83 | 92 |

### EXAMPLES 19-20

### Sustained Release Donepezil Formulations

| | mg/Tablet | |
|---|---|---|
| Ingredient | Example 19 | Example 20 |
| Donepezil HCl | 23 | 23 |
| Eudragit L 100 55 | 40 | 40 |
| Hypromellose K 100 LV | 35 | 35 |
| Sodium carboxymethyl cellulose | 15 | — |
| Sodium alginate | — | 15 |
| Lactose monohydrate | 65 | 65 |
| Hydroxypropyl cellulose (HPC-L) | 5 | 5 |
| Hydroxypropyl cellulose (HPC-EXF) | 5 | 5 |
| Magnesium stearate | 2 | 2 |
| Opadry White 58900 | 8.12 | 8.12 |
| Iron oxide red | 0.29 | 0.29 |
| Iron oxide yellow | 0.14 | 0.14 |

Manufacturing Process:

A) Sifting:

1. Lactose monohydrate is passed through an ASTM #20 mesh sieve.

2. Donepezil hydrochloride, Eudragit L 100 55, HPMC K100 LV, and sodium carboxymethyl cellulose or sodium alginate are sifted through an ASTM #20 mesh sieve and combined with step 1.

B) Granulation:

1. Hydroxypropyl cellulose is slowly added to water, with stirring to form a clear solution.

2. The mixture of A is granulated using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization at an inlet air temperature 50±10° C. for 30 minutes. LOD measured at 105° C. for 5 minutes is ≦3% w/w.

C) Milling:

1. Dried granules are milled through a Quadro co-mill fitted with 32R screen at 2500 rpm speed.

D) Blending and Lubrication:

1. Milled granules are blended for 5 minutes in a double cone blender.

2. Magnesium stearate and HPC EXF are sifted through an ASTM #30 mesh sieve, added to the double cone blender and blended for 7 minutes.

E) Compression:

1. The blend is compressed into 8 mm round tablets having hardness 10-14 Kp, thickness 3.9-4.3 mm, and friability 0.01%.

F) Film Coating:

1. Opadry White 58900 is mixed with iron oxide red and iron oxide yellow and slowly added into water under continuous stirring. The stirring is continued for 45 minutes until a smooth homogeneous suspension is obtained.

2. Compressed tablets are coated with the Opadry dispersion in a coating pan, using a spray rate 5 g/minute, pan speed 8 rpm, inlet temperature 60° C., and a weight gain of 4%. Coated tablets are cured at 50° C. for 20 minutes.

Samples of the tablets prepared in Examples 19 and 20 are tested for their drug dissolution profiles, and results are shown in Table 5. Dissolution is conducted in 900 mL of 0.1N HCl medium, using USP type 2 apparatus, stirred at 50 rpm for 14 hours.

TABLE 5

Cumulative % of
Drug Dissolved

| Hours | Example 19 | Example 20 |
|---|---|---|
| 1 | 32 | 30 |
| 2 | 51 | 47 |
| 3 | 64 | 61 |
| 4 | 74 | 72 |
| 5 | 82 | 81 |
| 6 | 88 | 87 |
| 8 | 93 | 94 |
| 10 | 94 | 97 |
| 12 | 95 | 98 |
| 14 | 96 | 99 |

### EXAMPLE 21-24

### Sustained Release Bi-Layer Donepezil Tablets

| | mg/Tablet | | | |
|---|---|---|---|---|
| | Example | Example | Example | Example |
| Ingredient | 21 | 22 | 23 | 24 |

Barrier Layer

| | | | | |
|---|---|---|---|---|
| HPMC K100 LV | 22 | — | 22 | — |
| Hydrogenated castor oil | — | 22 | — | 22 |
| Eudragit L 100 55 | 40 | 40 | 40 | 40 |
| Lactose monohydrate | 30 | 30 | 30 | 30 |
| Magnesium stearate | 2 | 2 | 2 | 2 |

Active Layer

| | | | | |
|---|---|---|---|---|
| Donepezil HCl | 23 | 23 | 23 | 23 |
| HPMC K100 LV | 22 | — | 22 | — |
| Hydrogenated castor oil | — | 22 | — | 22 |
| Lactose monohydrate | 39 | 39 | 39 | 39 |
| HPC-L | 5 | 5 | 5 | 5 |
| HPC-EXF | 5 | 5 | 5 | 5 |
| Magnesium stearate | 2 | 2 | 2 | 2 |

Coating

| | | | | |
|---|---|---|---|---|
| Opadry White 58900 | 8.12 | 8.12 | — | — |
| Iron oxide red | 0.29 | 0.29 | — | — |
| Iron oxide yellow | 0.14 | 0.14 | — | — |
| Eudragit L 100 55 | — | — | 25 | 25 |
| HPMC 3 cP | — | — | 25 | 25 |
| Triethyl citrate | — | — | 0.5 | 0.5 |
| Talc | — | — | 1 | 1 |

Manufacturing Process:

A) Sifting:

1. Active layer: Donepezil hydrochloride, HPMC K100 LV or hydrogenated castor oil, HPC-EXF, and lactose monohydrate are sifted through an ASTM #20 mesh sieve.

2. Barrier layer: Eudragit L 100 55, HPMC K100 LV or hydrogenated castor oil, and lactose monohydrate are sifted through an ASTM #20 mesh sieve.

B) Granulation:

1. Hydroxypropyl cellulose is slowly added to water, with stirring to form a clear solution.

2. The active layer and barrier layer ingredients are granulated separately using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization and an inlet air temperature 50±10° C. for 30 minutes. LOD measured at 105° C. for 5 minutes is ≦3% w/w.

C) Milling:

1. Dried granules are milled through an ASTM #24 mesh sieve.

D) Blending and Lubrication:

1. Milled granules are blended for 10 minutes in a double cone blender.

2. Magnesium stearate is sifted through an ASTM #30 mesh sieve.

3. The granules of active layer and barrier layer are lubricated separately with magnesium stearate in a double cone blender and blended for 7 minutes.

E) Compression:

1. Lubricated granules of active layer and barrier layer are compressed into bi-layer tablets.

F) Coating:

### EXAMPLES 21 AND 22

1. Opadry White 58900 is mixed with iron oxide red and iron oxide yellow and slowly added into water which is maintained under continuous stirring. Stirring is continued for 45 minutes until a smooth homogeneous suspension is obtained.

2. Compressed tablets are coated with the dispersion in a coating pan, using a spray rate 5 g/minute, pan speed 8 rpm, inlet temperature 60° C., and a weight gain of 4%. Coated tablets are cured at 50° C. for 20 minutes.

### EXAMPLES 23 AND 24

1. HPMC 3 cP is slowly added to water with continuous stirring. Stirring is continued for 45 mines to form a smooth homogeneous suspension.

2. Eudragit L 100 55 and triethylcitrate are slowly added sequentially to the suspension from step 1 under continuous stirring.

3. Talc is slowly added to the suspension from step 2 and stirred for 30 minutes.

4. Tablets are coated, using the procedure described for Examples 21 and 22, in step 2.

### EXAMPLES 25-28

### Sustained Release Tri-Layer Donepezil Tablets

| | mg/Tablet | | | |
|---|---|---|---|---|
| | Example | Example | Example | Example |
| Ingredient | 25 | 26 | 27 | 28 |

Barrier Layer 1

| | | | | |
|---|---|---|---|---|
| HPMC K100 LV | 11 | — | 11 | — |
| Hydrogenated castor oil | — | 11 | — | 11 |
| Eudragit L 100 55 | 20 | 20 | 20 | 20 |
| Lactose monohydrate | 15 | 15 | 15 | 15 |
| Magnesium stearate | 1 | 1 | 1 | 1 |

Active Layer

| | | | | |
|---|---|---|---|---|
| Donepezil HCl | 23 | 23 | 23 | 23 |
| HPMC K100 LV | 22 | — | 22 | — |
| Hydrogenated castor oil | — | 22 | — | 22 |
| Lactose monohydrate | 39 | 39 | 39 | 39 |
| HPC-L | 5 | 5 | 5 | 5 |
| HPC-EXF | 5 | 5 | 5 | 5 |
| Magnesium stearate | 2 | 2 | 2 | 2 |

Barrier Layer 2

| | | | | |
|---|---|---|---|---|
| HPMC K100 LV | 11 | — | 11 | — |
| Hydrogenated castor oil | — | 11 | — | 11 |
| Eudragit L 100 55 | 20 | 20 | 20 | 20 |
| Lactose monohydrate | 15 | 15 | 15 | 15 |
| Magnesium stearate | 1 | 1 | 1 | 1 |

Coating

| | | | | |
|---|---|---|---|---|
| Opadry White 58900 | 8.12 | 8.12 | — | — |
| Iron oxide red | 0.29 | 0.29 | — | — |
| Iron oxide yellow | 0.14 | 0.14 | — | — |
| Eudragit L 100 55 | — | — | 25 | 25 |
| HPMC 3 cP | — | — | 25 | 25 |
| Triethyl citrate | — | — | 0.5 | 0.5 |
| Talc | — | — | 1 | 1 |

Manufacturing Process:

A) Sifting:

1. Active layer: donepezil hydrochloride, HPMC K100 LV or hydrogenated castor oil, HPC-EXF, and lactose monohydrate are sifted through an ASTM #20 mesh sieve.

2. Barrier layers 1 and 2: Eudragit L 100 55, HPMC K100 LV or hydrogenated castor oil, and lactose monohydrate are sifted through an ASTM #20 mesh sieve.

B) Granulation:

1. Hydroxypropyl cellulose is slowly added to water, with stirring to form a clear solution.

2. The active layer and barrier layer powder mixtures are granulated separately using a fluid bed granulator with top spray. Granulation process parameters are inlet and exhaust temperatures at 60° C. and 35° C., respectively, product temperature 35° C., atomization air at 1 bar. The loaded material is pre-warmed to at least 45° C. and fluidized with air, then the solution is sprayed onto the powder.

3. Granules are dried with fluidization and an inlet air temperature 50±10° C. for 30 minutes. LOD measured at 105° C. for 5 minutes is ≦3% w/w.

C) Milling:

1. Dried granules are milled through a ASTM #24 mesh sieve.

D) Blending and Lubrication:

1. Milled granules are blended for 10 minutes in a double cone blender.

2. Magnesium stearate is sifted through an ASTM #30 mesh sieve.

3. The granules of the active layer and the barrier layers are lubricated separately with magnesium stearate in a double cone blender and blended for 7 minutes.

E) Compression:

1. Lubricated granules of active layer are compressed between the lubricated barrier layers, into a tri-layered dosage form.

F) Coating:

### FOR EXAMPLES 25 AND 26

1. Opadry White 58900 is mixed with iron oxide red and iron oxide yellow and slowly added into water with continuous stirring. The stirring is continued for 45 minutes until a smooth homogeneous dispersion is obtained.

2. Compressed tablets are coated with the dispersion in a coating pan, using a spray rate 5 g/minute, pan speed 8 rpm, inlet temperature 60° C., to a weight gain of 4%. Coated tablets are cured at 50° C. for 20 minutes.

For Examples 27 and 28:

1. HPMC 3 cP is slowly added to water with continuous stirring, and stirred for 45 minutes until a smooth homogeneous mixture is obtained.

2. Eudragit L 100 55 and triethyl citrate are slowly added sequentially to the mixture of step 1 with continuous stirring.

3. Talc is slowly added to the mixture from step 2 and stirred for 30 minutes.

4. The mixture of step 4 is maintained under stirring and used to coat tablets as described for Examples 25 and 26, in step 2.

1. A sustained release tablet formulation comprising donepezil or a pharmaceutically acceptable salt thereof, further comprising at least one release rate controlling material that is a hydrophilic material, hydrophobic material, enteric polymer, or any combination thereof.

2. The sustained release tablet formulation according to claim 1, wherein the concentration of release rate controlling material ranges from about 5 percent to about 90 percent by weight of the formulation.

3. The sustained release tablet formulation according to claim 1, wherein a hydrophilic material comprises a hydroxyalkyl cellulose, hydroxyalkyl alkyl cellulose, carboxyalkyl cellulose ester, cross-linked polyvinylpyrrolidone, or polyalkylene oxide.

4. The sustained release tablet formulation according to claim 1, wherein a hydrophobic material comprises a synthetic wax, vegetable wax, fatty alcohol, or a fatty acid or derivative thereof.

5. The sustained release tablet formulation according to claim 1, wherein an enteric polymer comprises a polyacrylate, methacrylic acid polymer, alginate, or a derivative thereof.

6. The sustained release tablet formulation according to claim 1, wherein a release rate controlling material comprises a hydrophilic material and an enteric polymer.

7. The sustained release tablet formulation according to claim 1, wherein a release rate controlling material comprises a hydrophilic material and a hydrophobic material.

8. The sustained release tablet formulation according to claim 1, wherein a release rate controlling material comprises a hydroxyalkyl cellulose or hydroxyalkyl alkylcellulose, together with a vegetable wax.

9. The sustained release tablet formulation according to claim 1, wherein a release rate controlling material comprises a hydroxyalkyl cellulose or hydroxyalkyl alkylcellulose, together with an enteric polymer.

10. The sustained release tablet formulation according to any of claim 1, wherein tablet matrix ingredients do not include an alkyl cellulose.

11. The sustained release tablet formulation according to claim 1, wherein a tablet matrix comprises a combination of a hydroxyalkyl alkylcellulose and a methacrylic acid polymer.

12. The sustained release tablet formulation according to claim 1, wherein a tablet matrix comprises a hydrophobic material and an enteric polymer, and wherein tablet matrix ingredients do not include an alkyl cellulose.

13. The sustained release tablet formulation according to claim 1, wherein a tablet matrix comprises a combination of a vegetable wax and a methacrylic acid polymer.

14. The sustained release tablet formulation according to claim 1, wherein a vegetable wax comprises a hydrogenated castor oil.

15. The sustained release tablet formulation according to claim 1, wherein tablets are prepared using wet granulation, dry granulation, or direct compression.

16. A sustained release formulation comprising a salt of donepezil, a hydrophilic material, and a wax material.

17. The sustained release formulation according to claim 16, wherein a hydrophilic material comprises a hydroxyalkyl alkylcellulose.

18. The sustained release formulation according to claim 16, wherein a wax material comprises a hydrogenated castor oil.

19. The sustained release formulation according to any of claim 16, in the form of a tablet having a coating comprising a hydrophilic or hydrophobic polymer.

20. A sustained release formulation comprising a salt of donepezil, a hydrophilic material, and an enteric polymer.

21. The sustained release formulation according to claim 20, wherein a hydrophilic material comprises a hydroxyalkyl alkylcellulose.

22. The sustained release formulation according to claim 20, wherein an enteric polymer comprises a methacrylic acid polymer.

23. The sustained release formulation according to any of claim 20, in the form of a tablet.
